(19) United States
(12) Patent Application Publication
(10) Pub. No.: US 2021/0199423 A1
(43) Pub. Date: Jul. 1, 2021

(54) METHOD FOR MEASURING HEIGHT OF SKIN BASAL CELL

(71) Applicant: NATIONAL TAIWAN UNIVERSITY, Taipei City (TW)

(72) Inventors: Chi-Kuang Sun, Taipei City (TW); Sheng-Tse Chen, Taipei City (TW)

(73) Assignee: NATIONAL TAIWAN UNIVERSITY, Taipei City (TW)

(21) Appl. No.: 16/726,953

(22) Filed: Dec. 26, 2019

(51) Int. Cl. G01B 11/06

(52) U.S. Cl. CPC G01B 11/0608

(57) ABSTRACT

The invention is related to a method for measuring the height of a skin basal cell, comprising the following steps: providing a non-invasive microscopy device for shooting a picture of at least one skin basal cell of skin to be tested, including a laser source for emitting laser light with pulsed laser and an image processing element for processing image signals; converging the laser light to the skin to be tested via the image processing element and obtaining 2D images; utilizing the image processing element to stack the 2D images; labelling the first appearing 2D image of the at least one basal cell as a first layer; labelling the just disappearing 2D image of the at least one basal cell as a last layer; and multiplying the stacking number of the stacking images between the first layer and the last layer with a preset distance between two stacking images via calculation, thereby obtaining the height of the at least one basal cell for assessing of a skin pigmentation patient to be cured.

### FIELD OF THE INVENTION

The invention relates to a method for measuring the height of a skin basal cell. Via a comparison of the height of a basal cell with skin disease and the height of a normal skin cell, assessment and analysis of pathological features are obtained.

### BACKGROUND ART

Pigment illness, such as sunburn, sacrum birthmark, etc., is a skin disease with extremely high prevalence and often onsets at the face portion and the hand portion of a human being. For a white older woman, the occurrence rate of sunburn on the face is up to ninety percent. As for an Asian older woman, the occurrence rate is up to seventy percent. A commonly seen treatment is a laser speckle surgery. However, after a certain time of speckle removal, almost all the surgery patient will relapse. Since the pigmented spot disease on the face is not with life threatening, the pathological research on such a disease is not complete, not to mention that the curative effect pathological assessment on the laser face treatment is for finding the cause of relapsing. Meeting with such a huge requirement for the medical and beauty market, the provisions of a picture beneath the skin with a micro pathology level in a high penetration, high resolution and quantification are badly needed.

### SUMMARY OF THE INVENTION

The invention is to improve the conventionally developed harmonic generation microscopy (HGM) by providing a method for measuring height of a skin basal cell for proceeding with synchronization and integration of a 3D facial recognition and diagnosis while in diagnosis with the doctor, with the aid of flexible orientation operation and better picture resolution and stability, so as to form a customized operation environment for picture processing to render the quantified pathological information regarding quantified melanin molecules, skin cells and tissues, etc.

Another objective of the invention is to provide a standard compatible with a transport agreement of the medical digital pictures, in which via clinical experiments of dermatology, the method is applicable to forensic and analysis of the pathological features of sunburn and sacrum nevus for long term tracing variation of after-operation lesion of the laser operation, thereby proceeding with curative effect pathological assessment before and after treating.

Further an objective of the invention is to provide an advanced method for optical virtual slice of a living body so as to proceed with overall non-invasive scanning and long term monitoring of the lesion area clinically, to facilitate improvement on grading of clinical disease, to provide policy determination for instant diagnosis and to engage in the curative effect assessment before and after treating.

In the method for measuring height of a skin basal cell, via the statistic result of the clinical experiments it may be found that the height of a skin basal cell is higher in a sunburn area than in a normal skin area, and such a sign exhibits with a notable difference in statistics. In addition, after an operation of laser spot removal, the height of the skin basal cell is reduced in the sunburn area, which represents a better curative effect. According to an intuition, the closer to the height of the normal skin basal cell, the better the curative effect.

According to the invention, it is provided a method for measuring the height of a skin basal cell, comprising the following steps:

providing a non-invasive microscopy device for shooting pictures of at least one skin basal cell of skin to be tested, including a laser source for emitting laser light with pulsed laser, and an image processing element for processing image signals;
converging the laser light to the at least one skin basal cell of the skin to be tested via the image processing element and obtaining 2D images by the image processing element;
utilizing the image processing element to stack the 2D images;
labelling the first appearing 2D image of the at least one skin basal cell as a first layer;
labelling the just disappearing 2D image of the at least one skin basal cell as a last layer; and
calculating a stacking number of the 2D stacking images between the first layer and the last layer and multiplying the stacking number of the 2D stacking images with a preset distance between two adjacent stacking images, thereby obtaining the height of the at least one skin basal cell for treatment assessing of a skin pigmentation patient to be cured,
wherein the stacking 2D images are of en face 2D images and are taken consecutively along an optical axis of the non-invasive microscopy device in a direction from skin epidermis to skin dermis, and the consecutive 2D images of the at least one skin basal cell are optionally chosen for a height assessment, and wherein an empty hole of one skin basal cell nuclei appears between the first layer and the last layer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing an upright skin basal cell measured according to the method for measuring the height of a skin basal cell according to the invention.

FIG. 2 is a schematic diagram showing a tilted skin basal cell measured according to the method for measuring the height of a skin basal cell according to the invention.

FIG. 3(a) is a block diagram showing an embodiment of a non-invasive microscopy device for use in the method for measuring the height of a skin basal cell according to the invention.

FIG. 3(b) is a block diagram showing a further embodiment of a non-invasive microscopy device for use in the method for measuring the height of a skin basal cell according to the invention.

FIG. 4 shows image diagrams of one skin basal cell obtained by using harmonic generation microscopy (HGM) according to the invention.

### DETAILED DESCRIPTION OF THE INVENTION

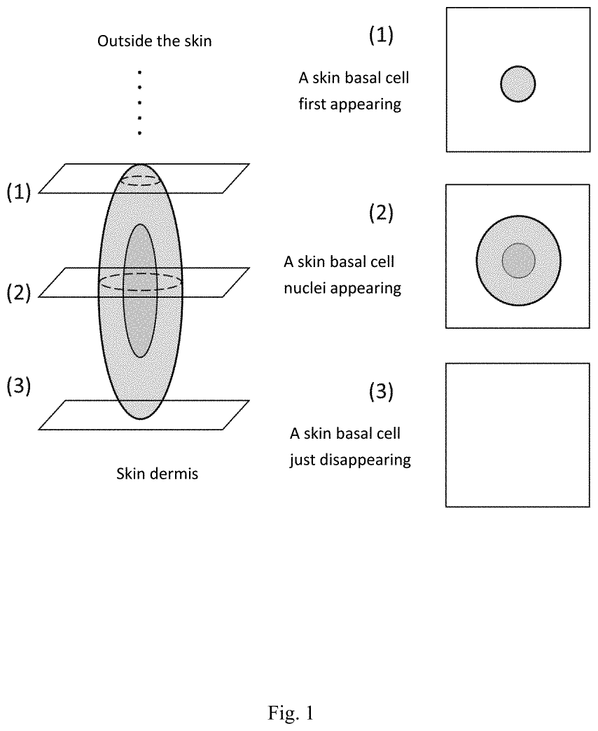

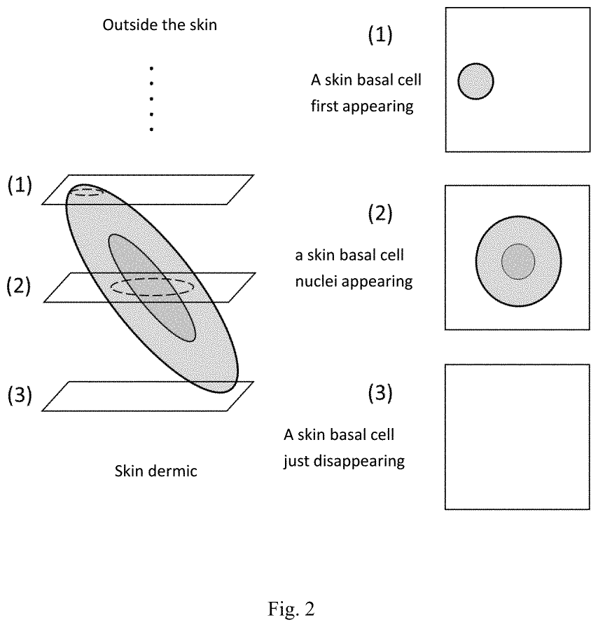

According to the method of analyzing a skin basal cell of the invention, FIG. 1, illustrating a schematic diagram of an upright skin basal cell, and FIG. 2, illustrating a schematic diagram of a titled skin basal cell, depict images of a skin basal cell along an en face direction in different depths, in which a first appearing image of a skin basal cell is a 2D image of (1), a just disappearing image of the skin basal cell is a 2D image of (3), and an image of (2) between (1) and (3) will appear an empty hole of the skin basal cell nuclei. As such, the distance between the two 2D images is the so-called a height of the skin basal cell.

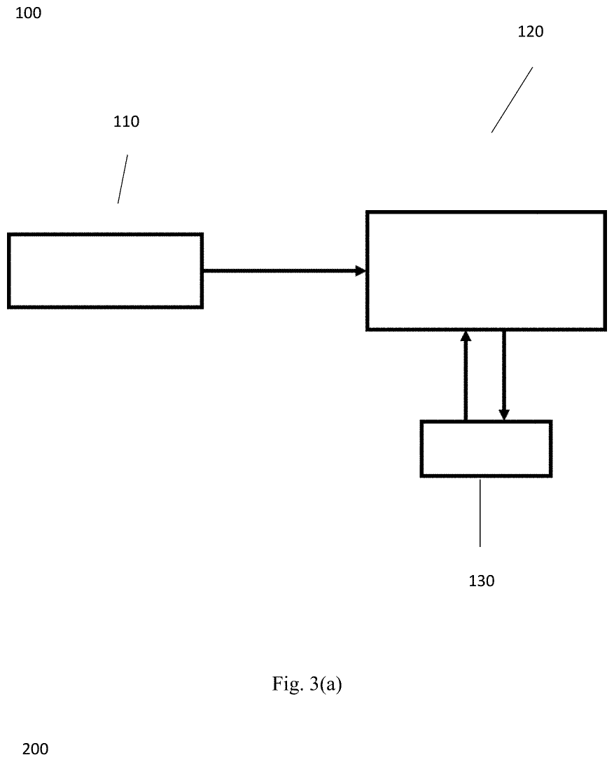

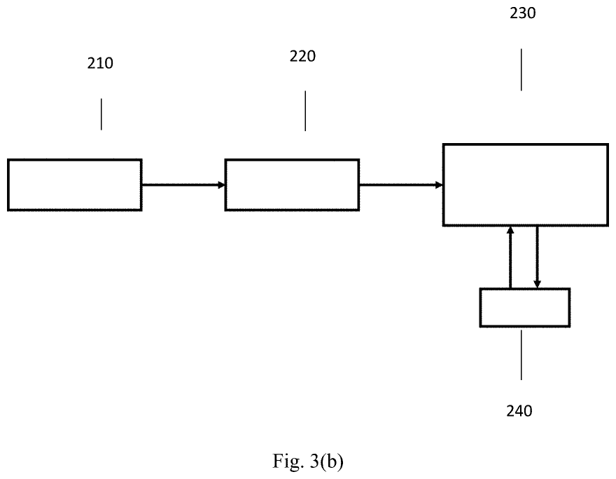

FIGS. 3(a) and 3(b) respectively show a structural block diagram of a different embodiment of a non-invasive microscopy device for use in the method for measuring the height of a skin basal cell according to the invention.

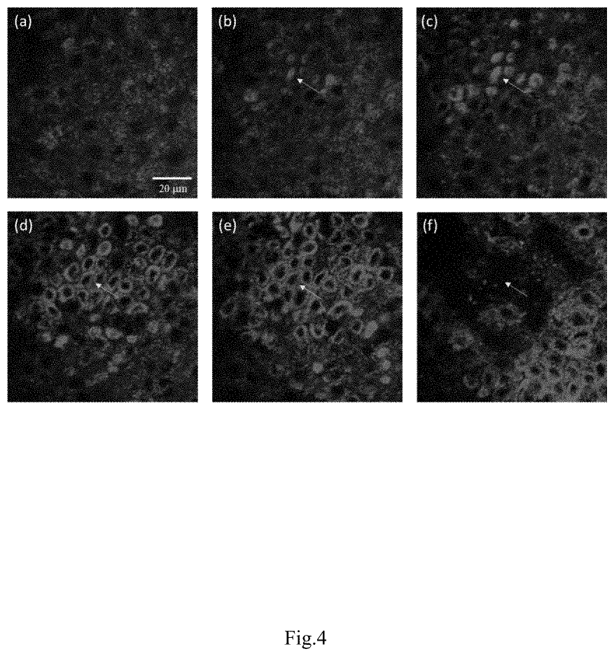

Please refer to FIG. 1, FIG. 2 and FIG. 3. It is proposed in the invention a method for measuring the height of a skin basal cell, comprising the following steps: providing a non-invasive microscopy device 100 for shooting pictures of at least one skin basal cell of skin to be tested 130, including, as shown in FIG. 3, a laser source 110 for emitting laser light with pulsed laser, and an image processing element 120 for processing image signals; converging the laser light to the at least one skin basal cell of the skin to be tested 130 via the image processing element 120 and obtaining 2D images by the image processing element; utilizing the image processing element 120 to stack the 2D images; labelling a first appearing 2D image, as indicated (1) in FIG. 1 and FIG. 2, of the at least one skin basal cell 130 as a first layer, also as indicated a layer (b) in FIG. 4; labelling a just disappearing 2D image, as indicated (3) in FIG. 1 and FIG. 2 of the at least one skin basal cell as a last layer, also as indicated a layer (f) in FIG. (4); and with reference to the image diagrams of one skin basal cell obtained by using the harmonic generation microscopy (HGM) as shown in FIG. 4, calculating a stacking number of the 2D stacking images between the first layer and the last layer, i.e. four layers between the layer (b) and the layer (f) as shown in the embodiment of FIG. 4, and multiplying the stacking number of the 2D stacking images with a preset distance between two stacking or two adjacent stacking images, in which the distance between the two stacking or adjacent images are the same, in which in one embodiment, the distance is set at 1.8 micrometers, and the height of the at least one skin basal cell for treatment assessing of a skin pigmentation patient to be cured is obtained by 1.8 (micrometer)×4 (stacking number)=7.2 micrometers, wherein the stacking 2D images are of en face 2D images and are taken consecutively along an optical axis of the non-invasive microscopy device in a direction from skin epidermis to skin dermis, and the consecutive 2D images of the at least one skin basal cell are optionally chosen for a height assessment, and wherein an empty hole of one skin basal cell nuclei appears between the first layer and the last layer, as indicated (2) in FIG. 1 and FIG. 2.

In the method for measuring the height of a skin basal cell in accordance with the invention, the non-invasive microscopy device 100 is a reflective confocal microscopy microscopy (RCM), two-photon excitation fluorescence microscopy (TPEF), harmonic generation microscopy (HGM) or optical coherence tomography (OCT).

Further, as shown in FIG. 3, another embodiment of a non-invasive microscopy device 200 used in the method for measuring the height of at least one skin basal cell of the invention includes a laser source 210, an image processing element 230 for shooting pictures of one skin basal cell 240, and in addition, a regulation means 220, disposed between the laser source 210 and the image processing element 230, for regulating a pulse width, strength and a path of the laser light, which comprises a double confocal mirror (DCM), a first lens, an attenuation sheet, a light guide, a reflective mirror, a 2D scanning Galvo system, a second lens, a splitter and a three-axis control platform, wherein the image processing element includes a convex lens, a splitter, two filters, two photomultiplier tubes and a computer, whereby, the images shot by such a structure of the non-invasive microscopy device may be double harmonic or triple harmonic microscopy.

In the method for measuring the height of a skin basal cell in accordance with the invention, in one example, the maximum diameter of the at least one skin basal cell along the en face direction is 10 micrometers, and a length and a width respectively of one whole piece of the image is larger than 10 micrometers and one whole piece of the image includes at least a skin basal cell. In addition, a horizontal resolution of the non-invasive microscopy device is smaller than 5 micrometers.

Further, a height of the skin basal cell is 20 micrometers and the Z-axis resolution of the non-invasive microscopy device is smaller than 10 micrometers, wherein the Z-axis of the non-invasive microscopy device is spaced smaller than 10 micrometers.

Furthermore, in the method for measuring the height of a skin basal cell in accordance with the invention, the at least one skin basal cell is in a plural number and the height of the plurality of the skin basal cells is in a mean value. In one example, the plurality of the skin basal cells are distributed in a Jiugongge grid.

What is claimed is:

1. A method for measuring the height of a skin basal cell, comprising the following steps:
providing a non-invasive microscopy device for shooting pictures of at least one skin basal cell of skin to be tested, including a laser source for emitting laser light with pulsed laser, and an image processing element for processing image signals;
converging the laser light to the at least one skin basal cell of the skin to be tested via the image processing element and obtaining 2D images by the image processing element;
utilizing the image processing element to stack the 2D images;
labelling a first appearing 2D image of the at least one skin basal cell as a first layer;
labelling a just disappearing 2D image of the at least one skin basal cell as a last layer; and
calculating a stacking number of the 2D stacking images between the first layer and the last layer and multiplying the stacking number of the 2D stacking images with a preset distance between two adjacent stacking images, thereby obtaining the height of the at least one skin basal cell for treatment assessing of a skin pigmentation patient to be cured,
wherein the stacking 2D images are of en face 2D images and are taken consecutively along an optical axis of the non-invasive microscopy device in a direction from skin epidermis to skin dermis, and the consecutive 2D images of the at least one skin basal cell are optionally chosen for a height assessment, and wherein an empty hole of one skin basal cell nuclei appears between the first layer and the last layer.

2. The method for measuring the height of a skin basal cell in accordance with claim 1, wherein the non-invasive microscopy device is a reflective confocal microscopy microscopy (RCM), two-photon excitation fluorescence microscopy (TPEF), harmonic generation microscopy (HGM) or optical coherence tomography (OCT).

3. The method for measuring the height of a skin basal cell in accordance with claim 1, further comprising a regulation means, disposed between the laser source and the image processing element, for regulating a pulse width, strength and a path of the laser light, and including a double confocal mirror (DCM), a first lens, an attenuation sheet, a light guide, a reflective mirror, a 2D scanning Galvo system, a second lens, a splitter and a three-axis control platform, wherein the image processing element includes a convex lens, a splitter, two filters, two photomultiplier tubes and a computer.

4. The method for measuring the height of a skin basal cell in accordance with claim 1, wherein the maximum diameter of the at least one skin basal cell on the en face direction is 10 micrometers.

5. The method for measuring the height of a skin basal cell in accordance with claim 4, wherein a length and a width respectively of one whole piece of the image is larger than 10 micrometers and the whole piece of the image includes at least one skin basal cell.

6. The method for measuring the height of a skin basal cell in accordance with claim 1, wherein a horizontal resolution of the non-invasive microscopy device is smaller than 5 micrometers.

7. The method for measuring the height of a skin basal cell in accordance with claim 1, wherein a height of the skin basal cell is 20 micrometers and a Z-axis resolution of the non-invasive microscopy device is smaller than 10 micrometers.

8. The method for measuring the height of a skin basal cell in accordance with claim 1, wherein Z-axis of the non-invasive microscopy device is spaced smaller than 10 micrometers.

9. The method for measuring the height of a skin basal cell in accordance with claim 1, wherein the at least one skin basal cell is in a plural number and the height of the plurality of the skin basal cells is in a mean value.

10. The method for measuring the height of a skin basal cell in accordance with claim 9, wherein the plurality of the skin basal cells are distributed in a Jiugongge grid.
